(19) United States
(12) Patent Application Publication
(10) Pub. No.: US 2024/0087691 A1
(43) Pub. Date: Mar. 14, 2024

(54) METHOD AND APPARATUS FOR PROCESSING SAMPLE

(71) Applicant: LEICA BIOSYSTEMS NUSSLOCH GMBH, Nussloch (DE)

(72) Inventors: Lingjun XIE, Shanghai (CN); Xiaoqi JIANG, Shanghai (CN); Gang SUN, Shanghai (CN); Fei WANG, Shanghai (CN)

(21) Appl. No.: 18/272,692

(22) PCT Filed: Jan. 25, 2021
(86) PCT No.: PCT/CN2021/073560

§ 371 Date: Jul. 17, 2023

(51) Int. Cl. G16H 10/40; B01L 9/00

(52) U.S. Cl. CPC G16H 10/40; B01L 9/52; B01L 2300/022; B01L 2300/0816

(57) ABSTRACT

This disclosure relates to a method and an apparatus for processing a sample. The method includes acquiring an identification of each cassette in a basket and an identification of each sample contained in the cassette by reading an electronic tag of the cassette, wherein the identification of the cassette and the identification of the sample contained in the cassette are stored in an associated manner in the electronic tag of the cassette; acquiring information of each sample from a library information system based on the identification of the sample; and recording the information of each sample for use in subsequent processing of the sample.

### CROSS-REFERENCE TO RELATED APPLICATION

The present application is a national phase entry under 35 USC § 371 of International Application PCT/CN2021/073560, filed Jan. 25, 2021, the entire disclosure of which is incorporated herein by reference.

### TECHNICAL FIELD

The present disclosure relates to a field of tissue processing technologies, and more particularly, to a method and an apparatus for processing a sample.

### BACKGROUND

In the tissue processing, sample tracking is important to users. Currently, a barcode is widely used to track the sample in a whole workflow for the tissue processing. However, since the sample typically is processed in a basket which usually contains more than 150 samples, it is difficult to identify all the samples by means of the barcode in short time, resulting in a long time for processing the samples. Therefore, it is desirable to provide a method to easily identify the samples to facilitate processing of the samples.

### SUMMARY

Embodiments of the present disclosure provide a method for processing a sample, an apparatus for processing a sample, an electronic device and a non-transitory computer-readable medium, which may identify a plurality of samples in short time so as to facilitate processing of the samples.

In a first aspect of embodiments of the present disclosure, a computer-implemented method for processing a sample is provided. The method may include acquiring an identification of each cassette in a basket and an identification of each sample contained in the cassette by reading an electronic tag of the cassette. The identification of the cassette and the identification of the sample contained in the cassette are stored in an associated manner in the electronic tag of the cassette. The method may further include acquiring information of each sample from a library information system based on the identification of the sample and recording the information of each sample for use in subsequent processing of the sample.

In an embodiment, the method may further include acquiring processing parameters for each sample during the subsequent processing; and writing the processing parameters for each sample into the electronic tag of the cassette containing the sample.

In an embodiment, each sample is integrated with an electronic tag. The method may further include for each sample, acquiring an identification of the sample by reading the electronic tag of the sample; acquiring the processing parameters for the sample by reading the electronic tag of the cassette containing the sample based on the identification of the sample; and writing the processing parameters for the sample into the electronic tag of the sample.

In an embodiment, the processing parameters include one or more of a temperature in the tissue processor, a pressure in the tissue processor, a concentration of a reagent for processing the sample, a kind of the reagent, a usage time of the reagent, and a number of cassettes and/or baskets and/or samples which have been processed in the reagent.

In an embodiment, the electronic tag of a cassette is attached to an outer surface of a body of the cassette or embedded in the body of the cassette.

In an embodiment, the electronic tag is a RFID tag.

In a second aspect of embodiments of the present disclosure, an apparatus for processing a sample is provided. The apparatus may include a first acquiring module, a second acquiring module and a recording module. The first acquiring module is configured to acquire an identification of each cassette in a basket and an identification of each sample contained in the cassette by reading an electronic tag of the cassette. The identification of the cassette and the identification of the sample contained in the cassette are stored in an associated manner in the electronic tag of the cassette. The second acquiring module is configured to acquire information of each sample from a library information system based on the identification of the sample. The recording module is configured to record the information of each sample for use in subsequent processing of the sample.

In an embodiment, the apparatus may further include a third acquiring module and a first writing module. The third acquiring module is configured to acquire processing parameters for each sample during the subsequent processing. The first writing module is configured to write the processing parameters for each sample into the electronic tag of the cassette containing the sample.

In an embodiment, each sample is integrated with an electronic tag. The apparatus may further include a fourth acquiring module, a fifth acquiring module and a second writing module. The fourth acquiring module is configured to, for each sample, acquire an identification of the sample by reading the electronic tag of the sample. The fifth acquiring module is configured to acquire the processing parameters for the sample by reading the electronic tag of the cassette containing the sample based on the identification of the sample. The second writing module is configured to write the processing parameters for the sample into the electronic tag of the sample.

In an embodiment, the processing parameters include one or more of a temperature in the tissue processor, a pressure in the tissue processor, a concentration of a reagent for processing the sample, a kind of the reagent, a usage time of the reagent, and a number of cassettes and/or baskets and/or samples which have been processed in the reagent.

In an embodiment, the electronic tag of a cassette is attached to an outer surface of a body of the cassette or embedded in the body of the cassette.

In an embodiment, the electronic tag is a RFID tag.

In a third aspect of the present disclosure, an electronic device is provided. The electronic device includes a processor and a memory having instructions executable by the processor stored therein. When the instructions are executed by the processor, the processor is caused to perform the above method according to any of embodiments of the present disclosure.

In a fourth aspect of the present disclosure, a non-transitory computer-readable medium is provided. The computer-readable medium includes instructions executable by a processor stored therein. When the instructions are executed by the processor, the processor is caused to perform the above method according to any of embodiments of the present disclosure.

With the method and the apparatus for processing a sample, the identification of the sample and the identification of the cassette containing the sample are stored in the electronic tag of the cassette, such that identification of each sample can be acquired by reading the electronic tag of the cassette containing the sample and then the information of the sample can be acquired to be used in the subsequent processing of the sample. In this way, the samples in a cassette can be easily identified in short time, such that the processing of the samples can be realized quickly.

### BRIEF DESCRIPTION OF THE DRAWINGS

In order to clearly illustrate technical solutions of embodiments of the present disclosure, a brief description of drawings used in embodiments is given below. Obviously, the drawings in the following descriptions are only part embodiments of the present disclosure, and for those skilled in the art, other drawings can be obtained according to these drawings without creative labor.

FIG. 1 is a flowchart of a method for processing a sample according to an embodiment of the present disclosure.

FIG. 2 is a schematic diagram of a cassette configured with an electronic tag according to another embodiment of the present disclosure.

FIG. 3 is a flowchart of a method for processing a sample according to another embodiment of the present disclosure.

FIG. 4 is a flowchart of a method for processing a sample according to a further embodiment of the present disclosure.

FIG. 5 is a block diagram of an apparatus for processing a sample according to an embodiment of the present disclosure.

FIG. 6 is a block diagram of an apparatus for processing a sample according to another embodiment of the present disclosure.

FIG. 7 is a block diagram of an apparatus for processing a sample according to a further embodiment of the present disclosure.

FIG. 8 is a block diagram of an electronic device according to an embodiment of the present disclosure.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the present disclosure. Instead, they are merely examples of apparatuses and methods consistent with aspects related to the present disclosure as recited in the appended claims.

In tissue processing, sample tracking is important to users. At present, the sample tracking is realized by means of a barcode of the sample. However, the sample is typically processed in the tissue processing by putting the sample in a basket which usually contains more than 150 samples, such that it is difficult to identify each sample in the basket in short time. Practically, there is a risk that some samples cannot be identified because the barcodes of the samples are blocked, which may cause failure to process the samples.

Accordingly, the embodiment of the present disclosure provides a method for processing a sample, an apparatus for processing a sample, an electronic device and a non-transitory computer-readable storage medium, to solve the problem to some extent.

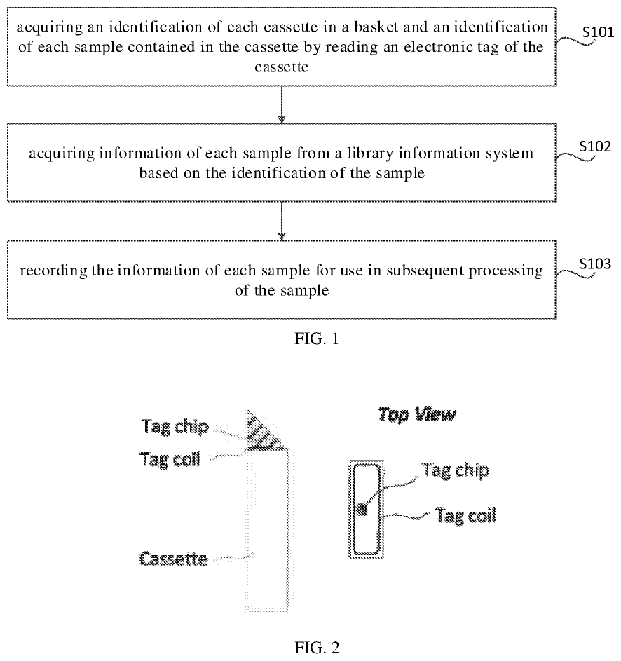

FIG. 1 illustrates a flowchart of a method for processing a sample according to an embodiment of the present disclosure. As illustrated in FIG. 1, the method includes the following steps.

At block S101, an identification of each cassette in a basket and an identification of each sample contained in the cassette are acquired by reading an electronic tag of the cassette.

The identification of the cassette and the identification of the sample contained in the cassette are stored in an associated manner in the electronic tag of the cassette.

After a sample is collected, an identification is defined for the sample. In the embodiment, the cassette is configured with an electronic tag which stores the identification of the cassette and the identification of each sample contained therein. The identification of the sample can be written into the electronic tag of the cassette if it is determined to put the sample in the cassette. By reading the electronic tag, the identification of each sample contained in the cassette can be acquired.

In some embodiments, the electronic tag of a cassette may be attached to an outer surface of a body of the cassette or embedded in the body of the cassette.

The electronic tag may be embedded in the body of the cassette or attached to the outer surface of the body of the cassette. As illustrated in FIG. 2, the electronic tag includes a tag chip and a tag coil. For example, the tag chip is embedded in the body of the cassette, and the tag coil is attached to the outer surface of the body.

In some embodiments, the electronic tag may be a RFID tag.

At block S102, information of each sample is acquired from a library information system based on the identification of the sample.

The library information system (LIS for short) is a system storing the identification and information of the sample. The information of the sample can be stored in LIS once the sample is collected. Further, the information of the sample can be supplemented as more information of the sample is determined.

After the identification of the sample is acquired, the information of the sample can be acquired from LIS based on the identification of the sample.

At block S103, the information of each sample in the cassette is recorded for use in subsequent processing of the sample.

In the embodiment, the information of each sample contained in the cassette may be recorded for use in the subsequent processing of the sample, for example, for tissue processing, embedding, sectioning and archiving. In this way, sampling tracking can be realized in the subsequent processing.

With the method for processing a sample, the identification of the sample and the identification of the cassette containing the sample are stored in the electronic tag of the cassette, such that identification of each sample can be acquired by reading the electronic tag of the cassette containing the sample and then the information of the sample can be acquired to be used in the subsequent processing of the sample. In this way, the samples in a cassette can be easily identified in short time, such that the processing of the samples can be realized quickly.

FIG. 3 illustrates a flowchart of a method for processing a sample according to another embodiment of the present disclosure. As illustrated in FIG. 3, the method includes the following steps.

At block S301, an identification of each cassette in a basket and an identification of each sample contained in the cassette are acquired by reading an electronic tag of the cassette.

At block S302, information of each sample is acquired from a library information system based on the identification of the sample.

At block S303, the information of each sample in the cassette is recorded for use in subsequent processing of the sample.

With regard to S301-S303, reference can be made to description of S101-S103 shown in FIG. 1, which will be not described herein.

At block S304, processing parameters for each sample are acquired during the subsequent processing.

During the subsequent processing of the sample, different processing parameters for processing the same sample may cause different results, or different processing parameters may be used for different samples, such that it is beneficial to record the processing parameters for each sample. The processing parameters for each sample during the subsequent processing can be acquired.

In some embodiments, the processing parameters include one or more of a temperature in the tissue processor, a pressure in the tissue processor, a concentration of a reagent for processing the sample, a kind of the reagent, a usage time of the reagent, and a number of cassettes and/or baskets and/or samples which have been processed in the reagent.

The usage time of the reagent and the number of cassettes and/or baskets and/or samples which have been processed in the reagent may affect the freshness of the reagent. For example, as the usage time of the reagent increases or the number of cassettes and/or baskets and/or samples which have been processed in the reagent increases, the freshness of the reagent may be decreased. If the freshness of the reagent is less than a predetermined value, the reagent cannot be used and should be replaced or updated, such that it is beneficial to record the factors such as the usage time of the reagent, the number of cassettes and/or baskets and/or samples which have been processed in the reagent that affect the freshness of the reagent.

At block S305, the processing parameters are written into the electronic tag of the cassette containing the sample.

In order to ensure a better sample tracking, the acquired processing parameters can be written into the electronic tag of the cassette containing the sample, such that the processing parameters of the sample can be acquired by reading the electronic tag of the cassette containing the sample as required.

In the embodiment, by acquiring the processing parameters of the sample and writing them into the electronic tag of the cassette containing the sample, the sample tracking can be realized in a better way.

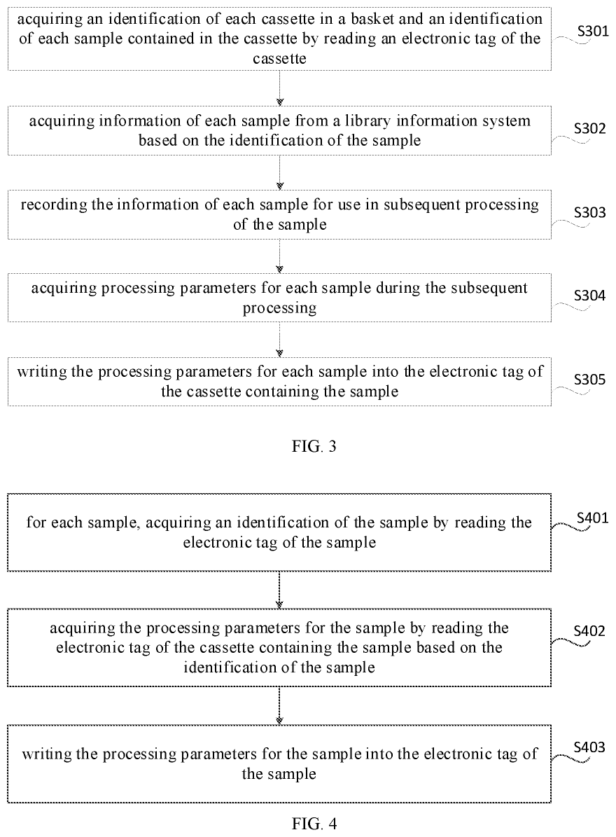

FIG. 4 illustrates a flowchart of a method for processing a sample according to another embodiment of the present disclosure. As illustrated in FIG. 4, based on embodiments described with reference to FIG. 3, the method may further include the following steps.

At block S401, for each sample, an identification of the sample is acquired by reading the electronic tag of the sample.

In the embodiment, each sample is configured with an electronic tag which stores the identification of the sample, such that the identification of each sample can be acquired by reading the electronic tag of the sample.

At block S402, the processing parameters for the sample are acquired by reading the electronic tag of the cassette containing the sample based on the identification of the sample.

Since the electronic tag of the cassette may record the processing parameters for each sample contained in the cassette and the identification of the sample, the processing parameters of a sample can be acquired by reading the electronic tag of the cassette based on the identification of the sample.

At block S403, the processing parameters for the sample are written into the electronic tag of the sample.

After acquiring the processing parameters of the sample, the processing parameters can be written into the electronic tag of the sample, such that the processing parameters for each sample can be acquired by reading the electronic tag of the sample even when the sample is taken from the cassette and the cassette is used to contain other samples subsequently.

In the embodiment, by acquiring the processing parameters of the sample and writing them into the electronic tag of the sample, the sample tracking can be realized even when the sample is used independent of the cassette.

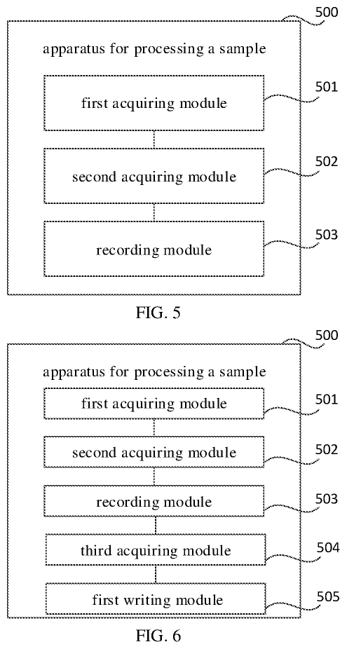

FIG. 5 is a block diagram of an apparatus 500 for processing a sample according to an embodiment of the present disclosure. As illustrated in FIG. 5, the apparatus 500 may include a first acquiring module 501, a second acquiring module 502 and a recording module 503.

The first acquiring module 501 is configured to acquire an identification of each cassette in a basket and an identification of each sample contained in the cassette by reading an electronic tag of the cassette. The identification of the cassette and the identification of the sample contained in the cassette are stored in an associated manner in the electronic tag of the cassette.

In an embodiment, the electronic tag of a cassette is attached to an outer surface of a body of the cassette or embedded in the body of the cassette.

In an embodiment, the electronic tag is a RFID tag.

The second acquiring module 502 is configured to acquire information of each sample from a library information system based on the identification of the sample.

The recording module 503 is configured to record the information of each sample for use in subsequent processing of the sample.

With the apparatus for processing a sample, the identification of the sample and the identification of the cassette containing the sample are stored in the electronic tag of the cassette, such that identification of each sample can be acquired by reading the electronic tag of the cassette containing the sample and then the information of the sample can be acquired to be used in the subsequent processing of the sample. In this way, the samples in a cassette can be easily identified in short time, such that the processing of the samples can be realized quickly.

FIG. 6 is a block diagram of an apparatus 500 for processing a sample according to another embodiment of the present disclosure. As illustrated in FIG. 6, based on the embodiments described with reference to FIG. 5, the apparatus 500 may further include a third acquiring module 504 and a first writing module 505.

The third acquiring module 504 is configured to acquire processing parameters for each sample during the subsequent processing.

The first writing module 505 is configured to write the processing parameters for each sample into the electronic tag of the cassette containing the sample.

In an embodiment, the processing parameters include one or more of a temperature in the tissue processor, a pressure in the tissue processor, a concentration of a reagent for processing the sample, a kind of the reagent, a usage time of the reagent, and a number of cassettes and/or baskets and/or samples which have been processed in the reagent.

In the embodiment, by acquiring the processing parameters of the sample and writing them into the electronic tag of the cassette containing the sample, the sample tracking can be realized in a better way.

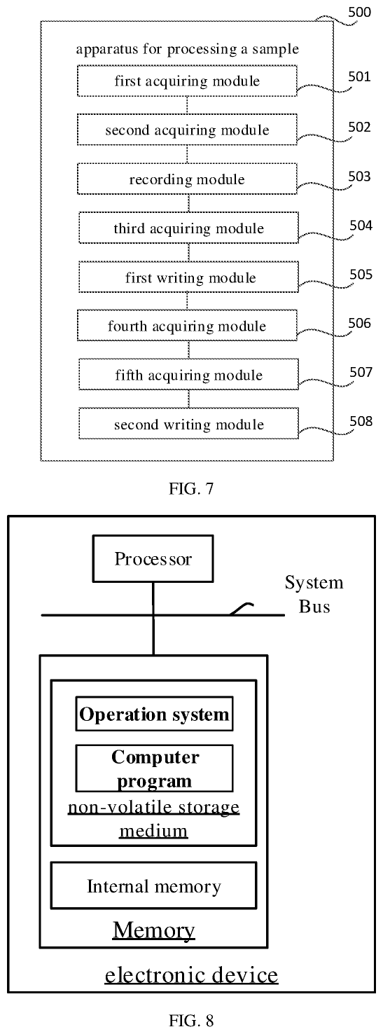

FIG. 7 is a block diagram of an apparatus 500 for processing a sample according to another embodiment of the present disclosure. As illustrated in FIG. 7, based on the embodiments described with reference to FIG. 6, each sample is integrated with an electronic tag, and the apparatus 500 may further include a fourth acquiring module 506, a fifth acquiring module 507 and a second writing module 508.

The fourth acquiring module 506 is configured to, for each sample, acquire an identification of the sample by reading the electronic tag of the sample.

The fifth acquiring module 507 is configured to acquire the processing parameters for the sample by reading the electronic tag of the cassette containing the sample based on the identification of the sample.

The second writing module 508 is configured to write the processing parameters for the sample into the electronic tag of the sample.

In the embodiment, by acquiring the processing parameters of the sample and writing them into the electronic tag of the sample, the sample tracking can be realized even when the sample is used independent of the cassette.

FIG. 8 is a schematic diagram of an internal structure of an electronic device according to an embodiment of the present disclosure. As illustrated in FIG. 8, the electronic device includes a processor and a memory connected by a system bus. The processor is used to provide computing and control capabilities to support operations of the entire electronic device. The memory may include a non-volatile storage medium and an internal memory. The non-volatile storage medium stores an operating system and computer programs. The computer program is executed by the processor to implement the method for processing a sample according to the embodiments of the present disclosure. The internal memory provides a cached operating environment for the operating system and the computer programs in the non-volatile storage medium. The electronic device may be a tissue processor.

Each module in the apparatus according to the embodiments of the present disclosure may be implemented in the form of a computer program. When the computer program is executed by the processor, the steps of the method according to the embodiments of the present disclosure are implemented.

The embodiments of the present disclosure also provide a computer-readable storage medium containing computer-executable instructions, when the computer-executable instructions are executed by the one or more processors, the one or more processors are caused to perform the steps of the method according to the embodiments of the present disclosure.

A computer program product containing instructions is provided. When the instructions are running on a computer, the computer is caused to perform the method according to the embodiments of the present disclosure.

Any reference to memory, storage, database, or other media used in this application may include non-volatile and/or volatile memory. Suitable non-volatile memory may include read-only memory (ROM), programmable ROM (PROM), electrically programmable ROM (EPROM), electrically erasable programmable ROM (EEPROM), or flash memory. Volatile memory can include random access memory (RAM), which is used as external cache memory. By way of illustration and not limitation, RAM is available in various forms, such as static RAM (SRAM), dynamic RAM (DRAM), synchronous DRAM (SDRAM), dual data rate SDRAM (DDR SDRAM), enhanced SDRAM (ESDRAM), synchronous Link (Synchlink) DRAM (SLDRAM), memory bus (Rambus) direct RAM (RDRAM), direct memory bus dynamic RAM (DRDRAM), and memory bus dynamic RAM (RDRAM).

The preferred embodiments of the present disclosure have been described in detail above in conjunction with the accompanying drawings; however, the present disclosure is not limited to the particular details in the above embodiments. Within the scope of technical concept of the present disclosure, various simple modifications may be made to the technical solutions of the present disclosure. These simple modifications all belong to the protection scope of the present disclosure.

In addition, it should be noted that the particular technical features described in the foregoing particular embodiments may be combined in any suitable manner without contradiction. In order to avoid unnecessary repetition, various possible combinations are not further explained in this disclosure.

In addition, various embodiments of the present disclosure can also be arbitrarily combined, as long as it does not violate the idea of the present disclosure, and it should also be regarded as the content disclosed in the present disclosure.

1. A computer-implemented method for processing a sample, comprising:
acquiring an identification of each cassette in a basket and an identification of each sample contained in the cassette by reading an electronic tag of the cassette, wherein the identification of the cassette and the identification of the sample contained in the cassette are stored in an associated manner in the electronic tag of the cassette;
acquiring information of each sample from a library information system based on the identification of the sample; and
recording the information of each sample on the electronic tag for use in subsequent processing of the sample.

2. The method of claim 1, further comprising:
acquiring processing parameters for each sample during the subsequent processing; and
writing the processing parameters for each sample into the electronic tag of the cassette containing the sample.

3. The method of claim 2, wherein each sample is integrated with an electronic tag, the method further comprising:
for each sample, acquiring an identification of the sample by reading the electronic tag of the sample;
acquiring the processing parameters for the sample by reading the electronic tag of the cassette containing the sample based on the identification of the sample; and
writing the processing parameters for the sample into the electronic tag of the sample.

4. The method of claim 2, wherein the processing parameters comprise one or more of a temperature in the tissue processor, a pressure in the tissue processor, a concentration of a reagent for processing the sample, a kind of the reagent, a usage time of the reagent, and a number of cassettes and/or baskets and/or samples which have been processed in the reagent.

5. The method of claim 1, wherein the electronic tag of the cassette is attached to an outer surface of a body of the cassette or embedded in the body of the cassette.

6. The method of claim 1, wherein the electronic tag is a RFID tag.

7. An apparatus for processing a sample, comprising:
a first acquiring module, configured to acquire an identification of each cassette and an identification of each sample contained in the cassette by reading an electronic tags of each cassette in a basket, wherein the identification of the cassette and the identification of the sample contained in the cassette are stored in an associated manner in the electronic tag of the cassette;
a second acquiring module, configured to acquire information of each sample from a library information system based on the identification of the sample; and
a recording module, configured to record the information of each sample for use in subsequent processing of the sample.

8. The apparatus of claim 7, further comprising:
a third acquiring module, configured to acquire processing parameters for each sample during the subsequent processing; and
a first writing module, configured to write the processing parameters for each sample into the electronic tag of the cassette containing the sample.

9. The apparatus of claim 8, wherein each sample is integrated with an electronic tag, the apparatus further comprising:
a fourth acquiring module, configured to, for each sample, acquire an identification of the sample by reading the electronic tag of the sample;
a fifth acquiring module, configured to acquire the processing parameters for the sample by reading the electronic tag of the cassette containing the sample based on the identification of the sample; and
a second writing module, configured to write the processing parameters for the sample into the electronic tag of the sample.

10. The apparatus of claim 8, wherein the processing parameters comprise one or more of a temperature in the tissue processor, a pressure in the tissue processor, a concentration of a reagent for processing the sample, a kind of the reagent, a usage time of the reagent, and a number of cassettes and/or baskets and/or samples which have been processed in the reagent.

11. The apparatus of claim 7, wherein the electronic tag of a cassette is attached to an outer surface of a body of the cassette or embedded in the body of the cassette.

12. The apparatus of claim 1, wherein the electronic tag is a RFID tag.

13. An electronic device, comprising:
a processor;
a memory, configured to store instructions executable by the processor, wherein when the instructions are executed by the processor, the processor is caused to perform the method according to claim 1.

14. A non-transitory computer-readable medium having instructions stored thereon for execution by a processor to implement operations of the method according to claim 1.
